(19) United States
(12) Patent Application Publication
(10) Pub. No.: US 2016/0374862 A1
(43) Pub. Date: Dec. 29, 2016

(54) COMPOSITE MATERIAL FOR FILLING CAVITY WOUNDS

(71) Applicants: VIVATECH, Paris (FR); LABORATOIRES URGO, Chenove (FR); HCP HEALTHCARE ASIA PTE.LTD, Singapour (SG)

(72) Inventor: Jean-Marc Pernot, Dijon (FR)

(73) Assignee: Urgo Recherche Innovation et Developpement, Chenove (FR)

(21) Appl. No.: 15/104,277

(22) PCT Filed: Dec. 19, 2014
(86) PCT No.: PCT/FR2014/053445

§ 371 Date: Jun. 14, 2016

(30) Foreign Application Priority Data
Dec. 20, 2013 (FR) 13 63156

(51) Int. Cl. A61F 13/00

(52) U.S. Cl. CPC A61F 13/00068; A61F 13/00021; A61F 13/00042; A61F 2013/00119

(57) ABSTRACT

The subject of the present invention is a composite wound packing material comprising a casing enclosing a material, or an assembly of materials, forming fluid flow channels, said casing being composed of a nonwoven material formed from a mixture of bicomponent superabsorbent fibers and non-absorbent thermal bonding fibers, said bicomponent superabsorbent fibers being of core/shell type, said core being made of polyacrylonitrile and the shell being made of polyacrylate.

The subject of the present invention is a composite wound packing material, especially for packing cavity wounds, which may be used in topical wound treatments which employ negative pressure devices.

In recent years, topical wound treatments which employ negative pressure therapy (NPT) devices have been developed considerably in the field of wound healing, due to their ability to accelerate the duration and quality of wound healing.

The basic principle of NPT treatments is to create a sealed cavity over the wound by means of a thin, flexible sealing film which is stuck to the skin of the patient surrounding the wound. The cavity also makes it possible to insert one end of a suction tube, the tube being, for example, sealed onto the sealing film and connected, at its other end, to a vacuum pump capable of creating, inside the cavity, a pressure lower than the ambient atmospheric pressure which surrounds the wound. The negative pressure created inside the cavity affords a number of beneficial therapeutic effects for wound healing, such as, especially, an increase in blood circulation and faster granulation of tissues. Different variants of these NTP treatments are currently in existence.

Topical wound treatments which employ negative pressure devices make it possible to treat different types of wounds, from the smallest lesions to the largest cavity wounds, or to burns, irrespective of their size. These injuries may also be deep and consequently have a large volume.

It is necessary to control the manner in which the wound heals. Ideally, the lesion should heal at depth to begin with, and then close up by joining together the edges of the wound in a uniform manner It is in particular desirable that the wound does not close up at the surface before the deep wound healing has finished, in order to avoid the formation of cavities in the flesh which would become favorable sites for infections. To avoid the formation of these cavities during treatment by NPT, the wound is generally packed by means of a soft or compressible porous material having properties which enable it to withstand the pressure difference created within the wound relative to the ambient atmospheric pressure. The purpose of the material is to hold the edges of the wound far enough apart that they cannot grow and join together to form an undesired cavity.

The material must also make it possible to provide fluid flow channels in order to ensure effective suction of exudates out of the wound, in general into a waste receptacle also referred to as a reservoir, connected to the suction tube.

The materials used in NPT systems are to be distinguished from conventional multilayer laminar dressings which aim to superficially protect small and substantially planar lesions and are not configured to be introduced within a wound, but simply to be positioned on the surface thereof so as to isolate it from the external environment while wound healing takes place. In particular, laminar dressings do not form fluid flow channels to enable effective drainage of exudates. In addition, the materials constituting these laminar dressings, in particular constituting the outer layer of these dressings, have a certain degree of adhesion to tissues and are not intended to be placed in contact with the wound. Use in a cavity wound would inevitably lead to contact between the material constituting the outer layer of the dressing and the tissues of the wound, which would cause an entirely unacceptable tearing out of budding tissue during removal of the dressing.

Most NPT systems sold at the current time use a material based on foam or gauze as wound packing material. However, porous materials of this type have the drawback of promoting tissue growth in their pores, which tissue growth attaches to said pores and, during removal of the material, this may damage the newly formed granulation tissue and be painful for the patient. In addition, such porous materials may also leave exudates in contact with the wound, causing an accumulation of bacteria, leading to infection.

Developments have thus been proposed in order to encase the porous materials of foam or gauze type by materials that are non-porous but that also do not stick to cutaneous tissues, in contact with which these materials are applied. International applications WO2009/071928 and WO2009/071938 from Smith and Nephew, especially, proposed composite wound packing materials comprising a non-porous casing enclosing a resilient material of foam or gauze type. The casing may additionally be coated or soaked in a non-stick gel such as a hydrogel or a silicone gel. In addition, it is also known practice to apply materials based on gelling fibers in contact with the wound, in planar form. These materials based on gelling fibers and which have the advantage of being non-stick are well known to those skilled in the art and are described for example in patent application WO2006/052839. By way of example of these materials based on gelling fibers, mention may especially be made of carboxymethyl celluloses (CMC) or salts thereof, alginates or else hyaluronic acid. These materials may well constitute the casing of the composite wound packing material. Nonetheless, the materials proposed in application WO2006/052839 have the drawback of separating from one another or delaminating under the effect of the pressure difference applied by the NPT. The use thereof as composite packing material wound therefore risk releasing debris into the would which could eventually become infected and/or disrupt the proper progression of the different steps of the wound healing mechanism.

There is therefore a real need to produce a composite wound packing material, especially for packing cavity wounds, having the desired properties of compressibility and resilience or deformability, while ensuring the flow of exudates without adhering to the cells of the wound, said composite material also having the ability to mechanically withstand the various mechanical stresses, such as the cycles of pressure applied by the NPT, without becoming destructured.

The subject of the present invention is thus a composite wound packing material comprising a casing enclosing a material, or an assembly of materials, forming fluid flow channels, said casing being composed of a nonwoven material formed from a mixture of bicomponent superabsorbent fibers and non-absorbent thermal bonding fibers, said bicomponent superabsorbent fibers being of core/shell type, said core being made of polyacrylonitrile and the shell being made of polyacrylate.

Within the context of the present invention, the term “composite wound packing material” is intended to mean a structure of core/shell type, the core consisting of a material, or an assembly of materials, forming fluid flow channels, the shell, also referred to as the casing, being composed of a nonwoven material formed from a mixture of bicomponent superabsorbent fibers and non-absorbent thermal bonding fibers.

### FIGURES

FIGS. 1A and B are explanatory diagrams of two embodiments of a wound packing material according to the invention, comprising a casing enclosing a material (FIG. 1A), or an assembly of materials (FIG. 1B), forming fluid flow channels (the flow of the exudates being represented, in a purely illustrative manner, by arrows in the figures).

FIG. 2 is a photograph of the assembly of the device used to carry out the mechanical strength test used in the examples according to the invention.

FIG. 3 is a photograph of the assembled device used to carry out the mechanical strength test used in the examples according to the invention.

FIG. 4 is a photograph of the Algostéril® product in the hydrated state after “1 cycle” of the mechanical strength test.

FIG. 5 is a photograph of the Aquacel® product in the hydrated state after “1 cycle” of the mechanical strength test.

FIGS. 6, 7A, 7B, 8A and 8B are photographs of the casing of the composite material according to different embodiments of the invention in the hydrated state, after “1 or 5 cycles”, depending on the case in question, of the mechanical strength test.

Nonwoven Casing Formed from a Mixture of Superabsorbent Fibers and Non-Absorbent Thermal Bonding Fibers

The composite wound packing material according to the invention comprises a casing being composed of a nonwoven material formed from a mixture of bicomponent superabsorbent fibers and non-absorbent thermal bonding fibers, said bicomponent superabsorbent fibers being of core/shell type, said core being made of polyacrylonitrile and the shell being made of polyacrylate.

According to one variant of the invention, said nonwoven may consist of a mixture of bicomponent superabsorbent fibers of core/shell type, said core being made of polyacrylonitrile and the shell being made of polyacrylate, and of non-absorbent thermal bonding fibers, all the fibers preferably being thermally bonded.

The nonwoven material especially does not stick to human tissues, and more particularly to the wound. Thus, during the removal thereof, in a dry or wet environment, but preferably in a wet environment, said nonwoven may be removed without the structure of the wound or of the perilesional skin being detrimentally affected.

The expression “superabsorbent” is intended to denote here fibers which have a very large capacity for absorbing liquids, preferably of greater than or equal to 10 g of water (or of saline solution, such as physiological saline) per gram, more preferably still of greater than 20 g of water per gram, and more preferably still of greater than 30 g of water per gram.

According to the invention, the superabsorbent fibers consist of two different materials. These materials may be distributed in a side-by-side configuration or preferably in a core/shell configuration.

The first material intended to form an outer part of the fiber, preferably the shell, should be able to form a gel with the wound exudates and will advantageously be formed from one or more crosslinked and/or partially crosslinked polymers. This first material is formed from polyacrylate.

The second component which will preferably form the core of the superabsorbent fibers will preferably be non-gelling and compatible with the first material, to guarantee the stability of the fiber after formation of a gel by the first material. It may be formed from any type of polymer which is stable in aqueous medium and compatible with the material of the shell, to give rise to a stable bicomponent fiber.

This second material is preferably formed from polyacrylonitrile.

The superabsorbent fibers advantageously have a decitex count of between 2 and 6 dtex.

Superabsorbent fibers which may be used within the context of the invention are for example sold by the company TOYOBO CO LTD under the name LANSEAL® F.

The non-absorbent fibers are thermal bonding fibers able to reinforce and stabilize the three-dimensional structure of the nonwoven by forming a framework resulting from the bonding of these fibers with one another, and/or of these fibers with the superabsorbent fibers.

These second fibers may consist of a single thermoplastic material, such as, for example, a polyethylene, a polypropylene or a polyester with a low melting point.

Advantageously, these second fibers will also consist of two different materials distributed in a side-by-side configuration or preferably a core/shell configuration.

The length of these fibers may be of the order of 10 to 100 mm, preferably 25 to 75 mm.

Within the context of the present invention, bicomponent non-absorbent thermal bonding fibers of core/shell type, in which the core is formed from a polyester, such as in particular polyethylene terephthalate, and the shell is formed from polyethylene, are particularly preferred.

The weight ratio between the superabsorbent fibers and the non-absorbent thermal bonding fibers may be between 20/80 and 80/20, preferably between 60/40 and 80/20.

Generally, the nonwoven constituting the casing of the composite material according to the invention will be obtained from mixtures incorporating more than 50% by weight, preferably more than 60% by weight, of superabsorbent fibers.

Excellent results were obtained by means of a mixture comprising 30% by weight of non-absorbent fibers and 70% by weight of superabsorbent fibers.

This nonwoven constituting the casing of the composite material according to the invention may especially be obtained by thermal bonding, or by needlepunching and thermal bonding of the mixture of fibers.

The thermal bonding operation makes it possible to improve the tear strength of the nonwoven after absorption, by creating anchoring points between the fibers of the nonwoven. It is necessary to reinforce the cohesion of the nonwoven to enable the removal of the used composite material without tearing it.

The assembling of the fibers will be carried out under conditions that make it possible to obtain a nonwoven having a thickness of between 0.3 and 3 mm, preferably of 2 mm, and a basis weight of between 30 and 400 g/m², preferably of the order of 100 g/m².

The nonwoven material constituting the casing of the composite material according to the invention may be manufactured according to the method described in document GB 2401879.

Contact Layer

According to one particular embodiment, and as long as this does not adversely affect the good cohesion properties of the composite material of the invention, the nonwoven casing may be partially covered with a contact layer on the face of the casing which is intended to come into contact with the wound, said layer comprising openings enabling the passage of wound exudates.

Advantageously, the contact layer is referred to as microadhesive, that is to say it makes it possible to temporarily affix said coated nonwoven to the wound. The assembly may then be removed without the structure of the wound or of the perilesional skin being adversely affected, so that said assembly is repositionable and facilitates nursing care. This temporary affixing can also assist the care personnel or the user in securing the dressing with other fixing means, e.g in covering the dressing with a support means or an adhesive tape. In this case, the contact layer may be chosen such that it has an adhesive strength on a steel plate of between 0.5 and 100 cN/cm, preferably of between 5 and 40 cN/cm. This adhesive strength is measured according to the method EN 1939, in which a sample of contact layer 20 mm wide and 150 mm long is placed on a steel plate and in which, after 10 minutes, the adhesive strength is measured with a dynamometer at a pull rate of 100 mm/min at a 90° angle.

The contact layer may preferably be formed from a composition comprising an elastomer matrix and hydrocolloids, and in particular an elastomer matrix in which hydrocolloids are preferably homogeneously dispersed.

The proportion of hydrocolloids is preferably between 2 and 20% by weight of the weight of said composition.

The contact layer may in particular cover between 55 and 65% of the face of the casing that is intended to come into contact with the wound.

The contact layer preferably has a basis weight ranging from 110 to 500 g/m², preferably from 150 to 200 g/m².

The contact layer advantageously makes it possible not to stick to the wound and to avoid any pain on removal of the composite wound packing material. By maintaining a moist environment at the surface of the wound while avoiding contact with the nonwoven casing, it improves wound healing. The incorporation of hydrocolloids gives the elastomer composition a hydrophilic nature and promotes the vectorization of active agents capable of promoting the treatment of the wound.

Said composition comprises one or more elastomers chosen from poly(styrene-olefin-styrene) block polymers. The block copolymers used within the context of the invention are advantageously triblock copolymers of ABA type comprising two styrene thermoplastic end blocks A and an elastomer central block B which is an olefin, optionally combined with diblock copolymers of AB type comprising a styrene thermoplastic block A and an elastomer block B which is an olefin. The olefin blocks B of these copolymers may consist of unsaturated olefins such as for example isoprene or butadiene or of saturated olefins such as for example ethylene-butylene or ethylene-propylene.

In the case of a mixture of triblock copolymers ABA and of diblock copolymers AB, it will be possible to use commercial mixtures of triblock copolymers ABA and of diblock copolymers AB that are already available or to produce mixtures in any proportion chosen beforehand from the two independently available products.

The triblock copolymers with an unsaturated central block are well known to those skilled in the art and are especially sold by Kraton Polymers under the name KRATON® D.

As examples of poly(styrene-isoprene-styrene) (abbreviated to SIS) copolymers, mention may thus be made of the products sold under the names KRATON® D1107 or KRATON® D1119 BT or else the products sold by Exxon Mobil Chemical under the name VECTOR® such as for example the product sold under the name VECTOR® 4113. An example of poly(styrene-butadiene-styrene) copolymers is the product sold under the name KRATON® D1102.

As examples of commercial mixtures of triblock copolymers ABA and of diblock copolymers AB in which B is isoprene, mention may be made of the products sold by Exxon Mobil Chemical under the name VECTOR® 4114.

All these copolymers based on isoprene or on butadiene generally have a styrene content of between 10% and 52% by weight relative to the total weight of said copolymer.

Within the context of the present invention, use will preferably be made of the poly(styrene-isoprene-styrene) (abbreviated to SIS) triblock block copolymers having a styrene content of between 14% and 52% and preferably of between 14% and 30% by weight relative to the weight of said poly(SIS).

Preferably, for producing the compositions of the present invention, use will be made of triblock block copolymers and in particular the product sold by Kraton Polymers under the name KRATON® D1119 BT.

The triblock copolymers having a saturated central block are also well known to those skilled in the art and are, for example, sold:

by Kraton Polymers under the name KRATON® G, and in particular under the name KRATON® G1651, KRATON® G1654 or KRATON® G1652 for poly(styrene-ethylene-butylene-styrene) (abbreviated to SEBS) block copolymers;

by Kuraray under the name SEPTON® for poly(styrene-ethylene-propylene-styrene) (abbreviated to SEPS) block copolymers.

As an example of commercial mixtures of triblock and diblock copolymers, mention may be made of the product sold by Kraton Polymers under the name KRATON® G1657, the olefin block of which is ethylene-butylene.

As an example of a particular mixture of triblock and diblock copolymers that can be produced within the context of the present invention, mention may be made of the mixture:

of a triblock SEBS, such as in particular the product sold by Kraton Polymers under the name KRATON® G1651; and

of a poly(styrene-olefin) diblock copolymer such as, in particular, the poly(styrene-ethylene-propylene) sold by Kraton Polymers under the name KRATON® G1702.

Within the context of the present invention, SEBS or SEPS triblock copolymers having a styrene content of between 25% and 45% by weight relative to the weight of said SEBS or SEPS will be preferred. Preferably, use will be made of triblock block copolymers and in particular the products sold by the company Kraton Polymers under the names KRATON® G1651 and KRATON® G1654.

Generally, the elastomer will be used in suitable amounts depending on the saturated or unsaturated nature of the olefin central block of the block copolymer. Thus, in the case of a triblock copolymer having an unsaturated central block it will be used in an amount of the order of 10% to 30% by weight, preferably of 10% to 20% by weight, relative to the total weight of the composition. In the case of a triblock copolymer having a saturated central block, it will be used in an amount of the order of 3% to 10% by weight, preferably of 4% to 7% by weight, relative to the total weight of the composition.

The term “hydrocolloid” or “hydrocolloid particles” is intended to mean here any compound customarily used by those skilled in the art for its ability to absorb aqueous liquids such as water, physiological saline or wound exudates.

As suitable hydrocolloids, mention may for example be made of pectin, alginates, natural vegetable gums such as, in particular, Karaya gum, cellulose derivatives such as carboxymethyl celluloses and the alkali metal salts thereof such as sodium or calcium salts thereof, and also synthetic polymers based on acrylic acid salts, known under the name “superabsorbents”, such as, for example, the products sold by BASF under the name LUQUASORB® 1003 or by Ciba Specialty Chemicals under the name SALCARE® SC91 and also mixtures of these compounds.

Some of these superabsorbents, described as “microcolloids” since they have a particle size of less than 10 micrometers, may of course be used within the context of the production of the composition.

The hydrocolloids preferred within the context of the present invention are the alkali metal salts of carboxymethyl cellulose, and in particular sodium carboxymethyl cellulose (CMC). The size of the hydrocolloid particles is for example between 50 and100 microns, especially of the order of 80 microns.

The amount of hydrocolloids incorporated into the elastomer composition will advantageously be of the order of 2% to 20% by weight, preferably of 5% to 18% by weight, more preferably still of 8% to 18% by weight, more preferably still of 12% to 16% by weight, relative to the total weight of the elastomer composition. Hydrocolloids introduced in too large an amount into a perforated contact layer reduce the absorption capacity of a nonwoven based on superabsorbent fibers as the gel forms. Indeed, the high absorption capacity of the hydrocolloids leads to a swelling of the contact layer, so much so that the holes of the mesh may become blocked. The nonwoven no longer directly absorbs the exudates but absorbs the exudates present in the hydrocolloid absorbent layer, which reduces the absorption capacity of the composite material and creates problems of maceration.

According to one preferred embodiment, the contact layer may comprise one or more elastomers chosen from the poly(styrene-olefin-styrene) block polymers combined with one or more plasticizing compound(s) intended to improve their stretching, flexibility, extrudability or processing properties.

They will preferably be liquid compounds, compatible with the olefin central block of the block copolymers used.

Among the plasticizing compounds capable of being used for this purpose, mention may in particular be made of plasticizing mineral oils, irrespective of the nature of the central block. Mention may also be made of polybutenes—such as, for example, the products sold by BP Chemicals under the name NAPVIS® 10—or else of phthalate derivatives such as dioctyl phthalate or dioctyl adipate, when the central block is unsaturated.

Alternatively, it is also possible to use synthetic products based on liquid mixtures of saturated hydrocarbons such as, for example, the products sold by Total under the name GEMSEAL® and in particular the product GEMSEAL® 60 which is an isoparaffinic mixture derived from a completely hydrogenated petroleum cut. Use will preferably be made of these products with a triblock copolymer comprising a saturated central block.

Within the context of the present invention, use will preferably be made of plasticizing oils and in particular of mineral oils formed from compounds of paraffinic, naphthenic or aromatic nature or mixtures thereof in variable proportions.

Among the plasticizing oils that are particularly suitable, mention may be made of:

the products sold by Shell under the names ONDINA® and RISELLA® which consist of mixtures based on naphthenic and paraffinic compounds;

the products sold under the name CATENEX® which consist of mixtures based on naphthenic, aromatic and paraffinic compounds.

Particularly preferably, use will be made of a mineral plasticizing oil chosen from the products sold under the names ONDINA®963 and ONDINA®919.

These plasticizing compounds may be used in an amount of the order of 20% to 65% by weight, preferably of 30% to 50% by weight, relative to the total weight of the hydrocolloid elastomer composition.

According to one embodiment, these compositions are referred to as adherent: they have the property of adhering to the skin without adhering to the wound. They comprise one or more compounds referred to as “tackifiers” such as those customarily used by those skilled in the art in the preparation of elastomer-based pressure-sensitive adhesives. For a detailed description of these products, reference may be made to the work by Donatas Satas “Handbook of Pressure Sensitive Technology”, 3rd Edition, 1999, pages 346 to 398.

Generally, it will be possible to use one (or more) tackifying product(s) which will be incorporated into the elastomer matrix in a proportion of the order of 1% to 50% by weight, relative to the total weight of the hydrocolloid elastomer composition, which will be determined as a function of the nature and of the relative proportion of the other constituents thereof, in order to achieve the desired micro-adhesive strength for the casing.

Preferably, the tackifying product(s) will represent from 10% to 45% by weight, and more preferably still from 15% to 40% by weight of the total weight of the hydrocolloid elastomer composition.

The tackifying products capable of being used within the context of the present invention will be able to be chosen from tackifying resins, low molecular weight polyisobutylenes or mixtures thereof.

Among the tackifying resins capable of being used according to the invention, mention may be made of modified terpene or polyterpene resins, rosin resins, hydrocarbon resins, mixtures of cyclic, aromatic and aliphatic resins, or mixtures of these resins.

Such products are sold, for example:

by Arakawa Chemical Industries under the name ARKON® P which are hydrogenated polycyclopentadiene resins;

by Exxon Chemical under the name ESCOREZ® and in particular the 5000 series of resins which are hydrogenated;

by Goodyear under the name WINGTACK®, and in particular WINGTACK® 86 which is a synthetic resin formed from C5/C9 copolymers or WINGTACK® 10 which is a resin based on synthetic polyterpene;

by the company Hercules under the name KRISTALEX® and in particular KRISTALEX® 3085 which is a resin based on α-methylstyrene.

Generally, in order to avoid coloring and stability problems of unsaturated resins, the use of hydrogenated resins, in particular with triblock copolymers having a saturated central block, is preferred since they are much more compatible with the latter than WINGTACK type unsaturated resins that are essentially used with triblock copolymers having an unsaturated central block.

Among the latter, use will preferably be made of ESCOREZ® resins of the 5000 series and most particularly the ESCOREZ® 5380 resin.

The tackifying resins may be used alone or as a mixture with other tackifying products, preferably in a proportion of 10% to 50% by weight, and more particularly of 15% to 40% by weight, relative to the total weight of the composition.

Among the low molecular weight polyisobutylenes capable of being used as tackifying products, mention may be made of the polyisobutylenes having a molecular weight of the order of 40 000 to 80 000 daltons, such as for example the products sold by BASF under the name OPPANOL® and in particular the products sold under the names OPPANOL® B12 and OPPANOL® B15 or by Exxon Chemical under the name Vistanex and in particular the LM-MH grade.

These polyisobutylenes will be able to be used alone or as a mixture with other tackifiers in combination with triblock copolymers having an unsaturated central block. Their proportion will be able to vary, in this case, from 5% to 30% by weight, and more particularly from 8% to 15% by weight, relative to the total weight of the composition.

Such contact layers are, for example, illustrated in application FR2973223.

The nonwoven casings formed from a mixture of superabsorbent fibers and non-absorbent thermal bonding fibers, partially covered with a contact layer on the face of the casing intended to come into contact with the wound, are especially sold under the name Urgoclean by Urgo.

Material, or Assembly of Materials, Forming Fluid Flow Channels

The composite wound packing material according to the present invention comprises a casing described above enclosing a material, or an assembly of materials, forming fluid flow channels, more particularly wound exudate flow channels.

The materials introduced into the casing of the composite material according to the invention may be porous or non-porous, compressible or non-compressible, deformable or non-deformable, resilient or non-resilient, as long as they perform their function of forming, intrinsically and/or by arranging them in relation to one another, fluid flow channels.

Within the meaning of the present application, “porous material” is intended to mean any material, the structure of which has cavities which may form fluid flow channels.

“Compressible material” is intended to mean any material, the volume of which decreases and the shape of which is modified under the effect of an external physical stress.

“Deformable material” is intended to mean any material, the shape of which is modified under the effect of an external physical stress, but the volume of which remains constant.

“Resilient material” is intended to mean any compressible or deformable material having the property of returning to its initial volume and/or its initial shape once the external physical stress has been removed. In other words, resilient material is intended to mean a material with shape memory.

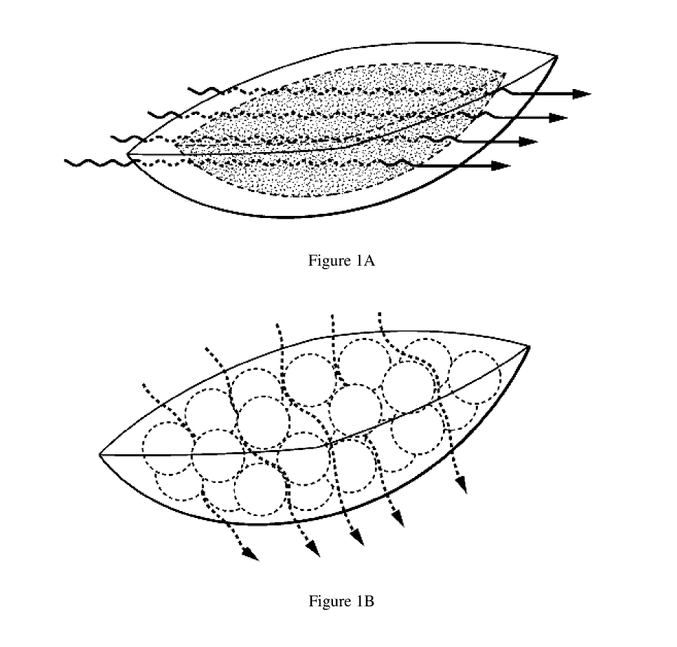

The composite material of the present invention consists of a “casing enclosing a material, or an assembly of materials”, that is to say it is in the form of a structure of core/shell type, the core consisting of a material, or an assembly of materials, forming fluid flow channels, the shell, also referred to as the casing, being composed of a nonwoven material formed from a mixture of bicomponent superabsorbent fibers and non-absorbent thermal bonding fibers, for example in the form of nonwoven alone or optionally combined with the contact layer described above. It should be noted that the shell completely encloses the material, or the assembly of materials, forming fluid flow channels. The material(s) forming fluid flow channels may have a greater or lesser mobility within the casing. Such structures are depicted schematically in FIGS. 1A and B.

According to a first preferred embodiment, the casing encloses a (single) material forming fluid flow channels. In this embodiment, the material is porous, compressible and resilient. The porosity of the material confers upon it the property of forming fluid flow channels intrinsically, due to the properties and the nature of the material used, having fluid flow channels within its structure.

According to this first embodiment, the casing may enclose a (single) material combining various properties, that is to say which may be porous or non-porous, compressible or non-compressible, deformable or non-deformable, resilient or non-resilient, as long as it performs its function of forming fluid flow channels intrinsically.

This first embodiment can be seen in FIG. 1A, in which the fluid flow channels allow exudates to pass through as illustrated in a schematic and purely illustrative manner by arrows in the figure.

According to a second preferred embodiment, the casing encloses an assembly of materials forming fluid flow channels.

In this second embodiment, the materials are separate from one another within the casing and separated by one or more interstices which may constitute fluid flow channels.

In this second embodiment, the materials may, according to a first preferred aspect, be porous, compressible and resilient. The fluid flow channels may then be formed both by the intrinsic porosity of the materials constituting the assembly, and by the interstices present between the different materials. Since the materials are compressible and resilient, and are also able to move with respect to one another within the casing, the assembly of these materials is also compressible and resilient.

Alternatively, in this second embodiment, the materials may, according to a second preferred aspect, be non-porous, non-compressible and non-deformable. The fluid flow channels are then formed solely by the interstices present between the different materials. The fluid flow channels allow exudates to pass through as illustrated in a schematic and purely illustrative manner by arrows in FIG. 1B. The non-compressible and non-deformable materials may move with respect to one another within the casing, thereby conferring upon the assembly of these materials a deformable character.

This second embodiment can be seen in FIG. 1B.

According to one specific alternative, it is entirely possible to envision providing a combination of said materials mentioned above. Thus, it is possible to combine, according to one embodiment of the invention, materials which are porous or non-porous, compressible or non-compressible, resilient or non-resilient and deformable or non-deformable, thus constituting an assembly of materials forming fluid flow channels. The intrinsic properties of each porous material, having inherent fluid flow channels because of its structure, and the properties conferred by an assembly of porous or non-porous materials, are of course retained in this type of combination. Thus, fluid flow channels may be formed both by the intrinsic porosity of a material and also by the interstices separating different materials arranged in an assembly, especially if the latter are able to move with respect to one another.

The porous, compressible and resilient material for filling the casing may, for example, comprise one or more foams or gauzes, but also any other suitable material having the required physical characteristics may be used as material for filling the casings. By way of example of porous, compressible and resilient material for filling the casing, mention may be made of polyurethane foams, foams based on poly(vinyl alcohol) or else based on cellulose, or on starch, or indeed various textile products based on synthetic or natural fibers chosen from the non-limiting list of compounds consisting especially of cotton, linen, wool, silk, chlorofibers, polyester, polyolefins, preferentially polyethylene, or else polyacrylic or polyamide fibers, in any form, whether thread, fiber, knit, woven, nonwoven or fabric, etc.

This material for filling the casing must be both compressible and also hard enough to hold the cutaneous tissues apart in the wound bed without being too aggressive for the tissues.

The porous material for filling the casing may thus have a hardness ranging from 5 to 100 Shore A and preferentially from 20 to 100 Shore A.

The non-porous, non-compressible and non-deformable materials for filling the casing may, for example, be chosen from PMMA (poly(methyl methacrylate)), glass, polystyrene, PVC, acrylonitrile butadiene styrene (ABS), silicone, SAN (styrene acrylonitrile), polyurethane, polyvinyl alcohol, cellulose, polyester, polyolefins, polyethylene, or a mixture of these materials. According to a preferred embodiment, the non-porous, non-compressible and non-deformable materials for filling the casing may be chosen from glass beads, polystyrene beads, silicone beads or polyethylene beads.

Active Agents

Various compounds may also be added to the casing and/or to the filling material of the composite materials of the present invention, such as, in particular, active agents or adjuvants commonly used in the field of wound treatment or in the pharmacological field.

The composite material may contain active principles that have a favorable role in wound treatment. These active principles may especially induce or accelerate wound healing. Other active agents may also be used within the context of the invention, such as, for example, bactericidal or bacteriostatic agents, antiseptics, painkillers or local anesthetics, anti-inflammatories, antipruritics, calmatives, hydrating agents, antioxidants, depigmenting agents and mixtures thereof.

Generally, these active agents may be chosen from:

active agents promoting wound healing, such as retinol, vitamin A, vitamin E, N-acetyl-hydroxyproline, Centella asiatica extracts, papain, silicones; thyme, niaouli, rosemary and sage essential oils; hyaluronic acid, allantoin, -Hema'tîte (Gattefossé), vitamin C, TEGO Pep 4-17(evonik), Toniskin (Silab), Collageneer (Expanscience), Timecode (Seppic), Gatuline skin repair (Gattefossé), panthenol, PhytoCellTec Alp Rose (Mibelle Biochemistry), Erasyal (Libragen), Serilesine (Lipotec), Heterosides of Talapetraka (Bayer), Stoechiol (Codif), Macarose (Sensient), Dermaveil (Ichimaru Pharcos), Phycosaccaride AI (Codify, growth factors, metformin, synthetic polysulfated oligosaccharides having 1 to 4 monosaccharide units, such as in particular sucrose octasulfate potassium salt (known by the abbreviation KSOS), sold in the form of the product Urgotul® Start by Laboratoires Urgo;

bactericidal or bacteriostatic agents such as polymyxin B, penicillins (amoxycillin), clavulanic acid, tetracyclines, minocycline, chlortetracycline, aminoglycosides, amikacin, gentamicin, neomycin, probiotics, silver salts such as silver sulfate, silver chloride, silver nitrate, silver sulfadiazine, quaternary ammoniums, polyhexamethylene biguanide and chlorhexidine;

antiseptics, such as thiomersal, eosin, chlorhexidine, phenylmercuric borate, aqueous hydrogen peroxide solution, Dakin's solution, triclosan, biguanide, hexamidine, thymol, Lugol's solution, iodinated povidone, merbromin, benzalkonium chloride, benzethonium chloride, ethanol or isopropanol;

painkillers or local anesthetics such as paracetamol, codeine, dextropropoxyphene, tramadol, morphine and its derivatives, or corticoids and derivatives;

anti-inflammatories, such as glucocorticoids, nonsteroidal anti-inflammatories, aspirin, ibuprofen, ketoprofen, flurbiprofen, diclofenac, aceclofenac, ketorolac, meloxicam, piroxicam, tenoxicam, naproxen, indomethacin, naproxcinod, nimesulide, celecoxib, etoricoxib, parecoxib, rofecoxib, valdecoxib, phenylbutazone, niflumic acid or mefenamic acid;

depigmenting agents, such as kojic acid (Kojic Acid SL®—Quimasso (Sino Lion)), arbutin (Olevatin®—Quimasso (Sino Lion)), the mixture of sodium palmitoylproline and of European white water lily extract (Sepicalm®—Seppic) or undecylenoylphenylalanine (Sepiwhite®—Seppic);

antipruritics: hydrocortisone, enoxolone, diphenhydramine, locally applied anti-H1 antihistamine;

moisturizing active agents, such as Xpermoist (Lipotec), hyaluronic acid, urea, fatty acids, glycerol, waxes or Exossine (Unipex);

UV-screening agents, such as Parsol MCX or Parsol 1789;

calmatives, such as camomile, bisabolol, xanthalene, glycyrrhetinic acid, tanactin (CPN) or Calmiskin (Silab);

antioxidants, such as vitamin E

According to a preferred embodiment, the active agents which may be introduced into the casing and/or into the filling material of the composite materials according to the present invention are preferably chosen from active agents which promote wound healing, anti-inflammatories and mixtures thereof.

“Active agent which promotes wound healing” is intended to mean any active agent capable of acting favorably at any stage of the wound healing process via any sort of interaction, that is to say via any interaction of biological, chemical or physical nature, with the wound in contact with which said active agent is applied.

More particularly, the active agents which may be introduced into the casing and/or into the filling material of the composite materials according to the present invention are preferably chosen from metformine, synthetic polysulfated oligosaccharides having 1 to 4 monosaccharide units, such as in particular sucrose octasulfate potassium salt, aspirin, silver sulfate, silver sulfadiazine and mixtures thereof.

Generally, the composite materials according to the present invention may comprise active agents in the casing and/or in the filling material at an amount of from 0.01 to 20% by weight, preferably from 1 to 15% by weight and more preferably still from 2 to 10% by weight, relative to the total weight of the casing and/or of the filling material containing them.

As adjuvants, mention may be made of dyestuffs, fillers, odor absorbers or trappers, pH regulators, microcapsules or microspheres that may optionally contain active agents, vaseline, polymers or surfactants making it possible to optimize the gelling rate, wettability or release of the active agents of the composite material.

The composite wound packing material according to the invention may be in any desired geometric shape, especially adapted to the shape and depth of the wound.

The casing is preferably closed around the filling material, or an assembly of filling materials, forming fluid flow channels, by heat sealing, by stitching or by one or more knots at the level of the casing, preferentially by heat sealing.

According to one particular embodiment, one or more porous, compressible and elastic filling materials may be introduced into the same casing.

According to one particular embodiment, the assembly of materials forming fluid flow channels may be in the form of a “pearl necklace”, that is to say that several filling materials forming fluid flow channels may be distributed individually in as many cavities of the nonwoven casing, separated from one another by heat sealing, by stitching or by one or more knots at the level of the casing, preferentially by heat sealing of said casing.

The present invention is illustrated in more detail in the following non-limiting example.

### EXAMPLE

Aim:

The mechanical strength (especially with regard to destructuring) of various composite wound packing materials was tested under conditions close to those of NPT (that is to say, under vacuum at 125 mmHg), in order to observe, among the various materials tested, which withstand the force exerted and which break.

The following apparatus and solution were used:

a MECA—004/SYN200 dynamometer, capable of applying a force of 26 N on contact with the desired material;

a 100N/MECA-008 sensor adjacent to the dynamometer;

a polished steel round-tipped metal rod of diameter 25.3866 mm and with circularity at the equator of 0.0093 mm;

an annular (gripping) clamp of internal diameter 44.4763 mm;

an NaCl/CaCl₂ solution comprising NaCl (8.298 g+/−5%) and Ca Cl₂ (0.368 g +−/5%).

The following materials constituting the casings of composite material according to the invention were tested:

a nonwoven with basis weight of 72 g/m² according to the present invention comprising bicomponent superabsorbent fibers of core/shell type, said core being made of polyacrylonitrile and the shell being made of polyacrylate;

a nonwoven with basis weight of 185 g/m² according to the present invention comprising bicomponent superabsorbent fibers of core/shell type, said core being made of polyacrylonitrile and the shell being made of polyacrylate;

a nonwoven with basis weight of 72 g/m² according to the present invention comprising bicomponent superabsorbent fibers of core/shell type, said core being made of polyacrylonitrile and the shell being made of polyacrylate, coated with a contact layer prepared according to the method described below;

a nonwoven with basis weight of 185 g/m² according to the present invention comprising bicomponent superabsorbent fibers of core/shell type, said core being made of polyacrylonitrile and the shell being made of polyacrylate, coated with a contact layer prepared according to the method described below;

the product sold under the name Aquacel® by Convatec®, composed of 100% gelling fibers consisting of sodium carboxymethyl cellulose;

the product sold under the name Algosteril® by Laboratoires Brothier® and consisting of gelling fibers of calcium alginate type.

A hydrophobic crosslinked polyurethane foam sold by AQF® under the trade name PDQZ30 was used as material forming fluid flow channels.

A contact layer was prepared according to the following protocol.

Moreover, a hydrocolloidal elastomer composition was prepared by mixing in a MEL G-40 blender.

The elastomer composition, expressed in percentage by weight relative to the total weight of the composition, was as follows:
mineral oil sold by Shell under the name Ondina®919: 41.7%;
(hydrocolloidal) carboxymethylcellulose sodium salt sold by AQUALON under the name CMC Blanose®7H4XF: 14.8%;
elastomeric poly(styrene-ethylene-butylene-styrene) block copolymer sold by KRATON under the name KRATON® G 1651 E: 4.7%;
antioxidant sold under the name IRGANOX® 1010 by CIBA SPECIALTY CHEMICALS: 0.2%;
tackifying resin sold by EXXON CHEMICALS under the name ESCOREZ® 5380: 35.6%;
copolymer of 2-methyl-2[(1-oxo-2-propenyl)amino]-1-propanesulfonic acid salt and of the 2-hydroxyethyl ester of propenoic acid sold by SEPPIC under the name SEPINOV® EMT 10: 5%.

The various constituents were introduced at a temperature between 105° C. and 115° C. with stirring, so as to obtain a homogeneous mixture. More specifically, initially the mineral oil, the hydrocolloid, and the elastomer, then the antioxidant, the releasing agent and finally the tackifying resin were introduced.

This adhesive was coated onto the nonwoven with basis weight of 72 g/m² and the basis weight of 185 g/m² at a basis weight of 180 g/m²±40 g/m² in the form of a net, the mesh of which is square. The coating is carried out by hot melt transfer on an etched cylinder. The thread thickness is 1.6 mm.

Operating Procedure

Force to Apply to the Samples

The NPT systems are regulated in the most common way to apply a vacuum of 125 mmHg. This corresponds to the application of a force of 26 N to the composite wound packing material.

Sample Preparation

3 square samples with 80 mm side lengths were cut using a punch from the AQF PDQZ30 foam constituting the material for filling the casing.

1 square sample of 80 mm side length was also cut using a punch from each of the nonwovens constituting the casing to be tested.

In parallel, a test solution comprising NaCl (8.298 g+/−5%) and CaCl₂ (0.368 g +−/5%) was prepared. This solution makes it possible, on the one hand, to simulate the moisture conditions found within a wound and, on the other hand, to simulate the saline concentration of the exudates found in a wound.

Finally, the squares of materials constituting the casing to be tested were soaked in the test solution for 30 minutes at 37° C.+2° C. After 30 minutes, the squares were removed and left to drip dry for approximately 30 seconds.

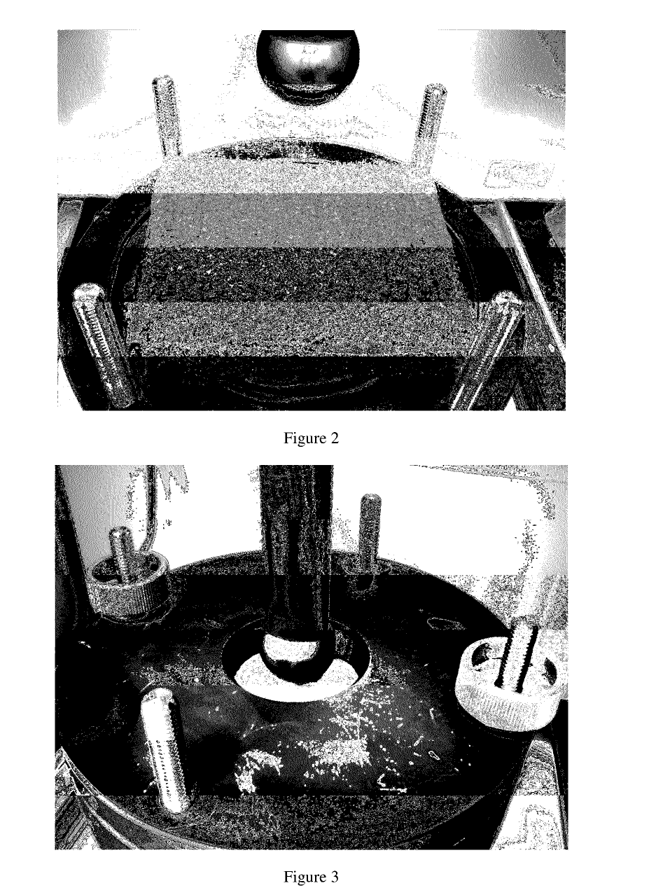

Each hydrated square was then superposed on a square of foam (which is not hydrated), then the composite material obtained in this way was positioned in the test device. FIG. 2 illustrates the test device on which the sample of composite material consisting of the superposed square of foam and nonwoven was deposited.

An annular gripping clamp is then affixed to the sample consisting of the composite material and is fixed by means of 4 clamping screws so as to ensure complete cohesion between the two materials. FIG. 3 illustrates a photo of the device after putting the gripping clamp in place.

The dynamometer is regulated such that the vertical falling speed of the round-tipped metal rod corresponds to (300+10) mm/min and such that this fall is stopped when the round-tipped metal rod applies a force of 26 N after having come into contact with the sample. After contact and application of the desired pressure to the sample, the round-tipped metal rod stops and rises back up.

Next, the resistance of the composite samples to this first cycle is observed.

If the sample has withstood this first cycle, the “rise and fall” cycle of the round-tipped metal rod is reproduced 5 times on the sample, said round tip applying a force of 26 N to the sample at each cycle.

Results:

The table below summarizes the state of the samples of composite material after 1 or 5 test cycles:

| Products tested | Results |
|---|---|
| Sample using the product Algostéril ® | Destructuring of the product: |
| (1 cycle) | round tip penetrates entirely |
| | through the Algostéril. |
| Sample using the product Aquacel ® | Destructuring of the product. |
| (1 cycle) | After contact between the |
| | round tip and the sample, the |
| | moist gelling material |
| | constituting the casing |
| | penetrates into the foam and |
| | remains partially trapped |
| | therein. |
| Sample using the nonwoven (72 g/m²) | No breaking of the product. |
| according to the invention (1 cycle) | |
| Sample using the nonwoven (72 g/m²) | No breaking of the product. |
| according to the invention (5 cycles) | |
| Sample using the nonwoven (185 g/m²) | No breaking of the product. |
| according to the invention (1 cycle) | |
| Sample using the nonwoven (185 g/m²) | No breaking of the product. |
| according to the invention (5 cycles) | |
| Sample using the nonwoven (72 g/m²) | No breaking of the product. |
| coated with the contact layer according to | |
| the invention (1 cycle) | |
| Sample using the nonwoven (72 g/m²) | No breaking of the product. |
| coated with the contact layer according to | |
| the invention (5 cycles) | |
| Sample using the nonwoven (185 g/m²) | No breaking of the product. |
| coated with the contact layer according to | |
| the invention (1 cycle) | |
| Sample using the nonwoven (185 g/m²) | No breaking of the product. |
| coated with the contact layer according to | |
| the invention (5 cycles) | |

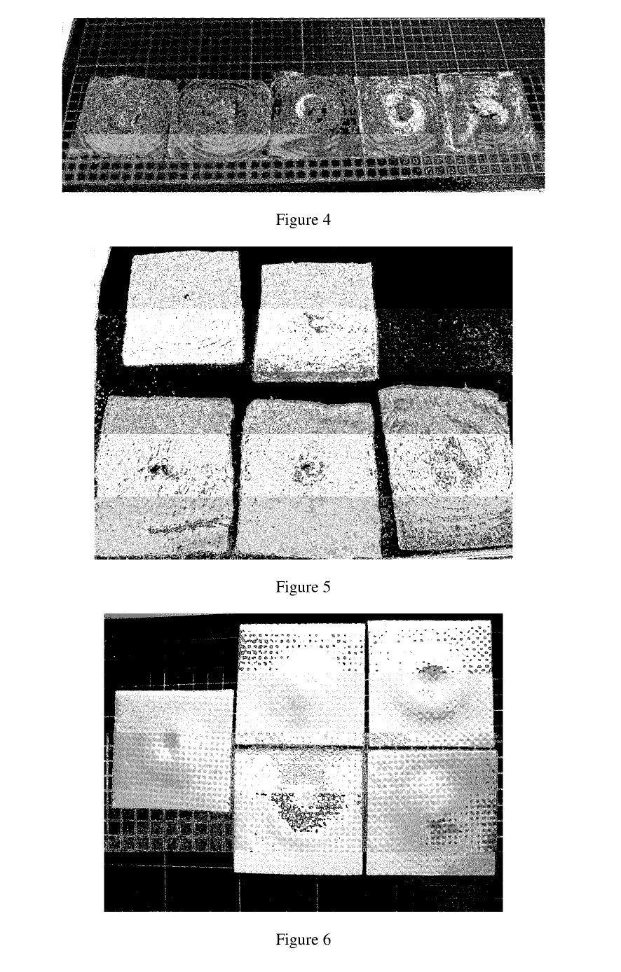

FIG. 4 illustrates the sample using the product Algostéril® after one test cycle. This product is entirely destructured following the pressure applied by the device simulating the pressure forces applied in NPT.

FIG. 5 illustrates the sample using the product Aquacel® after one test cycle. This product is entirely destructured following the pressure applied by the device simulating the pressure forces applied in NPT.

FIG. 6 illustrates the sample using the nonwoven of basis weight 72 g/m² coated with the contact layer according to the invention, that is to say a nonwoven comprising bicomponent superabsorbent fibers of core/shell type, said core being made of polyacrylonitrile and the shell being made of polyacrylate, the whole thing being coated with the contact layer as defined above, after 5 test cycles. It is noted that the product undergoes only very slight deformations, and in no case does it become either entirely or partially destructured.

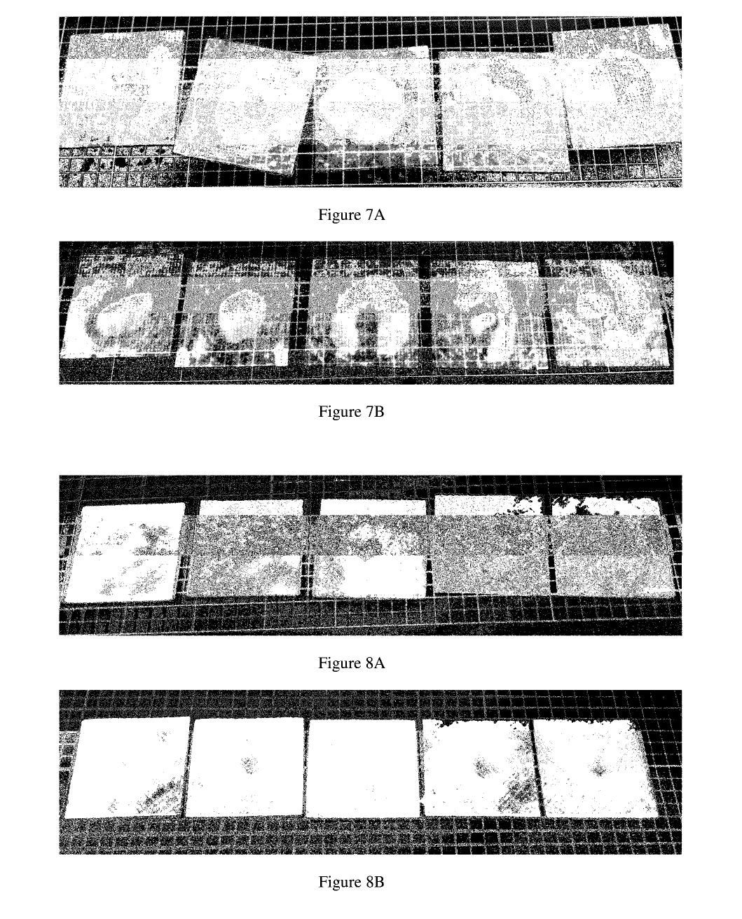

FIG. 7 illustrates the sample using the nonwoven of basis weight 72 g/m² according to the invention, that is to say a nonwoven comprising bicomponent superabsorbent fibers of core/shell type, said core being made of polyacrylonitrile and the shell being made of polyacrylate, after 1 (FIG. 7A) and 5 (FIG. 7B) test cycles, respectively. It is noted that the product undergoes only very slight deformations, and in no case does it become either entirely or partially destructured.

FIGS. 8A and B illustrate the sample using the nonwoven (185 g/m²) according to the invention, that is to say a nonwoven comprising bicomponent superabsorbent fibers of core/shell type, said core being made of polyacrylonitrile and the shell being made of polyacrylate, after 1 (FIG. 8A) and 5 (FIG. 8A) test cycles, respectively. It is noted that the product does not become either entirely or partially destructured.

The sample using the nonwoven according to the invention is therefore the only one which may be used as composite material having the desired properties of compressibility and resilience or deformability, while ensuring the flow of exudates without adhering to the cells of the wound, said composite material also having the ability to mechanically withstand the various mechanical stresses, such as pressure or the cycles of pressure applied during NPT, without becoming destructured.

1. A composite wound packing material comprising a casing enclosing a material, or an assembly of materials, forming fluid flow channels, said casing being composed of a nonwoven material formed from a mixture of bicomponent superabsorbent fibers and non-absorbent thermal bonding fibers, said bicomponent superabsorbent fibers being of core/shell type, said core being made of polyacrylonitrile and the shell being made of polyacrylate.

2. The composite wound packing material of claim 1, wherein the non-absorbent thermal bonding fibers of the casing are bicomponent, said bicomponent being of core/shell type, said core being made of polyethylene terephthalate and the shell being made of polyethylene.

3. The composite wound packing material as claimed in either one of the preceding claims, characterized in that the nonwoven material has a basis weight ranging from 30 to 400 g/m².

4. The composite wound packing material of claim 1, wherein the materials introduced into the casing may be porous or non-porous, compressible or non-compressible, deformable or non-deformable, resilient or non-resilient, as long as they perform their function of forming fluid flow channels.

5. The composite wound packing material as of claim 1, wherein the casing encloses a material forming fluid flow channels, said material being porous, compressible and resilient.

6. The composite wound packing material of claim 1, wherein the casing encloses an assembly of materials forming fluid flow channels.

7. The composite wound packing material of claim 1, wherein the materials are porous, compressible and resilient.

8. The composite wound packing material of claim 1, wherein the materials are non-porous, non-compressible and non-deformable.

9. The composite wound packing material of claim 4, wherein the porous, compressible and resilient material(s) for filling the casing comprise one or more foams or gauzes, and/or have a hardness ranging from 5 to 100 Shore A and preferentially from 20 to 100 Shore A.

10. The composite wound packing material of claim 5, wherein the porous, compressible and resilient material(s) for filling the casing comprise one or more foams or gauzes, and/or have a hardness ranging from 5 to 100 Shore A and preferentially from 20 to 100 Shore A.

11. The composite wound packing material of claim 6, wherein the porous, compressible and resilient material(s) for filling the casing comprise one or more foams or gauzes, and/or have a hardness ranging from 5 to 100 Shore A and preferentially from 20 to 100 Shore A.

12. The composite wound packing material of claim 7, wherein the porous, compressible and resilient material(s) for filling the casing comprise one or more foams or gauzes, and/or have a hardness ranging from 5 to 100 Shore A and preferentially from 20 to 100 Shore A
